(19) United States
(12) Patent Application Publication
(10) Pub. No.: US 2026/0307297 A1
(43) Pub. Date: Oct. 8, 2026

(54) Silicate Fiber Polymer Composite

(71) Applicant: Tundra Composites, LLC, White Bear Lake, MN (US)

(72) Inventors: Adam Bartel, St. Paul, MN (US); Scott Bohnen, Stillwater, MN (US); Kurt Heikkila, Marine on the St.Croix, MN (US)

(73) Assignee: Tundra Composites, LLC, White Bear Lake, MN (US)

(21) Appl. No.: 19/713,923

(22) Filed: Jun. 19, 2026

Related U.S. Application Data
(63) Continuation of application No. 18/529,516, filed on Dec. 5, 2023.
Application No. 18/529,516 is a continuation of application No. 17/525,235, filed on Nov. 12, 2021, now abandoned.

(51) Int. Cl. B29B 9/14; A61B 17/17; A61B 17/80; A61B 17/88; B29C 48/00; C08K 7/10; C08K 9/04; C08L 27/06

(52) U.S. Cl. CPC B29B 9/14; A61B 17/1728; A61B 17/8019; A61B 17/8052; A61B 17/808; A61B 17/888; A61B 17/8891; B29C 48/00; C08K 7/10; C08K 9/04; C08L 27/06; B29C 2948/92123; B29C 2948/92142; C08K 2201/003; C08K 2201/004; C08K 2201/016; C08L 2203/30

(57) ABSTRACT

The claimed material relates to a silicate fiber and polymer composite having enhanced modulus, viscoelastic and rheological properties.

### CROSS-REFERENCE TO RELATED APPLICATIONS

This application claims the benefit of a U.S. Patent Provisional Application Ser. No. 62/755,740, filed Nov. 5, 2018. This application is hereby incorporated by reference in its entirety.

### FIELD

Disclosed is a composite of a silicate fiber and a polymer. The thermoplastic composite has improved processing characteristics, improved structural product properties that produce enhanced products. The novel properties are produced in the composite by novel interactions of the coated fiber components and polymer components.

### BACKGROUND

Composite materials have been made for many years by combining generally two dis-similar materials to obtain beneficial properties from both. In a true composite, the interactions of the component materials provide the best properties and characteristics of both components. The use of a reinforcing fiber produces a range of materials and, under the correct conditions, can form a polymer composite. In contrast, a simple filled polymer, following the rule of mixtures, with additive or filler, displays composite properties that are simply expected based on the nature of the materials. Fillers are often used as simple replacements for an expensive component to reduce costs in the composition. We do not consider filled polymers to be a composite. Also, strongly coupled or covalently bonded materials, such as polymer to fiber or fiber to fiber, have minimal viscoelastic properties.

Substantial attention has been paid to the creation of composite materials with unique properties. Fiber and fabric reinforced polymer materials can include cellulosic fiber, high modulus polyolefin fiber, polybenzoxazole fiber, carbon fiber, aramid fiber, boron fiber, glass fiber and hybrid materials. The fiber can be used in reinforcing thermoplastics and thermosets. Epoxy and polyurethane thermosets are common. Polyolefin, polyvinyl chloride (PVC), polyester, epoxy, polyurethane, and other thermoplastics or thermosets and hybrids have been developed for a variety of end uses.

Developing thermoplastic composite materials have faced difficult barriers. To obtain significant thermal processing, tensile, modulus, impact and COTE properties, a composite needs to control the degree of interaction between reinforcing fiber and polymer and the degree of fiber loading in the polymer matrix. Highly filled composite polymer materials, grater than 40 vol. %, cannot be easily made. Melt processing thermoplastics and fibers are not easily combined due to interactions with the polymer with respect to uncoated fiber composition, fiber roughness, surface energy and fiber morphology. Excessive compounding processing to obtain a uniform composite can cause fiber damage and thermal depolymerization of the polymer with accompanying hazards of fire and the release of toxic gasses. While fiber composites have been proposed with high fiber loadings, commercial products have not achieved much greater than about 40-50 vol. % fiber.

While a substantial amount of work has been done regarding fiber reinforced thermoplastic polymer composite materials. A substantial need exists for a composite material that has a fiber surface that is made more inert and more compatible with respect to the polymer, improved fiber packing efficiency and with high fiber content, improved thermal compounding processing that maintains fiber integrity with improved rheology, and to produce composites with improved structural properties at elevated use temperatures.

### BRIEF DESCRIPTION

A composite of an interfacially modifier (IM) coated silicate fiber and a polymer has improved and novel properties. The claimed composite is made of a combination of a thermoplastic polymer and an interfacial modified silicate (not silica (e.g.) glass) fiber. The composite can be made of about 10 to 90 wt. % of a continuous phase comprising the polymer with about 90 to 10 wt. % of a discontinuous s phase comprising the fiber. The composite properties result from a selection of fiber composition, length, diameter and aspect ratio, polymer type, molecular weight, viscoelastic character and processing conditions. The resulting composite materials exceed the contemporary structural composites in packing, surface inertness, processability, COTE, tensile properties modulus and physical modulus. In the process of making the composite, the fiber input to the compounding process unit can have an arbitrary length, often about 0.8 to 100 mm. The product output of the compounding process unit can have a fiber of similar length, depending on process conditions. The fiber can be reduced in length if sheared in compounding. The composite containing the fiber can be pelletized. In the pellet, the fiber cannot be longer than the major dimension of the pellet. The pellet can be formed into any product shape needed.

One aspect of the claimed material is a composite of interfacially modified coated silicate fiber and polymer. Such a composite material has unique rheological and strength properties and is a thermoplastic in character and can be extruded and then melted and reextruded.

One aspect of the claimed material is a composite of interfacially modified wollastonite fiber and polymer. The fibers can be densified. Such a composite material has unique rheological and strength properties and is a thermoplastic composite that can be extruded and then melted and reextruded.

An aspect is in a pellet made of the composite can be used as an intermediate article between the compounding of the composite and the manufacturing of the final product. Such a pellet can comprise the composite comprising the components designed to be directly used in making an article. Alternatively, the pellet can comprise a master batch composition with increased amounts of fiber (typically greater than 50 vol. %) such that the pellet can be combined with additional polymer in proportions that result in producing use concentrations.

Another aspect is a structural member made of the composite. Such structural members can be used in commercial and residential construction and can include decking, siding and fenestration units including trim, windows and doors.

A final aspect of the claimed material is a method of compounding the composite by compounding the combined polymer resin and interfacial modified fiber under melt processing conditions.

As used in this disclosure the term “fiber” means a collection of similar fibers in a fibrous material combined with a polymer as input to a compounding process unit. Fiber as used in a discontinuous phase can be free of a particle or particulate.

Particle, or a collection of particles known as a particulate, is a discrete object having a particle size about 0.1-500 microns, an aspect ratio of less than 3 and a circularity ((circularity, is measured by a view of the two-dimensional projection of a particle and is equal to (perimeter)²/area)) is less than 20.

As used in this disclosure the term “continuous phase” means the polymer matrix into which the fiber is dispersed during compounding.

As used in this disclosure the term “discontinuous phase” means the individual fibers or particles that are dispersed throughout the continuous phase.

As used in this disclosure the term “interfacial modifier” means a material that can coat the surface of fiber and does not react or interact with the polymer or other coated fiber present in the composite. In one embodiment, the interfacial modifier material is an organo-metallic material.

Silica is SiO₂ which is not a silicate. Silicate is a reaction product of silica and a basic reactant and is resident in a negatively charged anionic species.

The term “densified” when used as a composite fiber material characteristic means a fiber source that is processed to increase the bulk density of the material such that is approaches the density of the polymer used in the composite. The initial density can be increased by at least a factor of 2 or about 3 to 5. Non-densified wollastonite silicate fiber that is naturally 0.2 to 0.4 g-cc⁻³ density is processed to be at least 0.4 g-cc⁻³ or 5 g-cc⁻³ or more.

### BRIEF DISCUSSION OF FIGURES

FIGS. 1-6 Show the results of testing Example 9 for its physical properties.

### DETAILED DISCUSSION

Novel composites are made by combining an interfacial modified fiber and a polymer to achieve novel physical and process properties. The composite can be made of about 10 to 90 wt. % of a continuous phase comprising the polymer with about 90 to 10 wt. % of a discontinuous phase comprising the fiber.

The fiber can comprise a metal (e.g., calcium or magnesium) silicate or meta silicate fiber through a reaction of a metal oxide with silica. While silicate chemistry is often complex, with varied propositions and crystal forms of metal oxide and silica. For example, calcium silicate can be expressed as CaSiO₃, Ca₂SiO₄ or 2CaO·SiO₂. A useful silicate fiber is wollastonite, a unique natural silicate (CaSiO₃) fiber containing small amounts of iron, magnesium and manganese. One aspect of the mineral can be represented as compositions including CaSiO₃—MgSiO₃·FeSiO₃ with the iron and magnesium components in minor proportions. Wollastonite is a crystalline fiber with chains of linked negatively charged SiO₄ tetrahedra.

Wollastonite is commonly used in ceramics, tiles, paints compositions. Wollastonite has been proposed as a substitute for asbestos in a variety of products but its unique inorganic nature has limited its applicability in thermoplastic composites. The morphology, roughness, surface energy and hydrophobic nature of the wollastonite fiber's uncoated surface severely limits commercial manufacture due to resulting poor melt polymer mixing, minimal packing and nonuniform composites. The use of a densified material can increase the process characteristics of the material and obtain improved product uniformity and physical properties. Without a uniform composite, the compounding processing and resulting mechanical properties can be lacking. Each of the individual fibers of the generic, useful fiber material has a cross-section dimension (preferably but not limited to a diameter) of at least about 0.8 micron often about 1-150 microns and can be 2-100 microns, or 5 to 10 microns, a length of 0.1-150 mm, often 0.2-100 mm, and often 50 to 100 mm and can have an aspect ratio of at least 50 often about 100-1500. These aspect ratios are typical of the input into the compounder. After pellets are formed the aspect ratio is limited by the pellet dimensions. A useful fiber is a wollastonite fiber as follows:

| Typical Properties: | VANSIL WG | | VANSIL HR-2000 | | VANSIL HR-1500 | |
|---|---|---|---|---|---|---|
| Density | 21.7 | lbs.-gal⁻¹ | 21.7 | lbs.-gal⁻¹ | 21.7 | lbs.-gal⁻¹ |
| | 2.9 | g-cc⁻¹ | 2.9 | g-cc⁻¹ | 2.9 | g-cc⁻¹ |
| pH, 10% slurry | 10-11 | | 10-11 | | 10-11 | |
| (ASTM D 1208) | | | | | | |
| G. E. Brightness | 86-88 | | 93-95 | | 93-95 | |
| (TAPPI T-646) | | | | | | |
| Bulk Density (loose) | 31 | lbs.-ft⁻³ | 25 | lbs.-ft⁻³ | 24 | lbs.-ft⁻³ |
| Bulk Density (tapped) | 45 | lbs.-ft⁻³ | 39 | lbs.-ft⁻³ | 37 | lbs.-ft⁻³ |
| Average Aspect Ratio | 15:1 | | 12:1 | | 14:1 | |
| Average Needle Length | 90 | μm | 65 | μm | 60 | μm |
| Average Needle Width | 9 | μm | 7 | μm | 5 | μm |
| Particle Size (Horiba LA-300): | | | | | | |
| D10 | 4 | μm | 4 | μm | 3 | μm |
| D50 | 28 | μm | 17 | μm | 13 | μm |
| D90 | 117 | μm | 65 | μm | 50 | μm |
| D95 | 146 | μm | 88 | μm | 68 | μm |
| Wet Screen Analysis: | | | | | | |

| | | | |
|---|---|---|---|
| Plus 100 mesh | <2% | 0.1% | <0.1% |
| Plus 200 mesh | 15-20% | <3% | <1% |
| Plus 325 mesh | 30-35% | ≤20% | 5-7% |

The fiber is typically coated with an interfacial surface chemical treatment also called an interfacial modifier (FM) that supports or enhances the final properties of the composite such as viscoelasticity, rheology, high packing fraction, and fiber surface inertness. These properties are not present in contemporary thermoplastic composite materials. An interfacially modified fiber has a substantially complete coating over the fiber surface of an interfacial modifier (IM) with a thickness of less than 1500 Angstroms (Å) often less than 200 Angstroms (Å), and commonly 10 to 500 Angstroms (Å) or 100 to 1500 Angstroms (Å).

A composite is more than a simple admixture with such properties that can be predicted by the rule of mixtures. A composite is defined as a combination of two or more substances at various percentages, in which each component results in providing properties of the composite material that are in addition to or superior to those of its separate constituents. In a simple admixture, the mixed materials have minimal property enhancement. In a composite material, at least one of the materials can be chosen to increase physical properties such as stiffness, strength or density. In the claimed composites, we seek to minimize or control VDW forces between fiber and polymer matrix and reduce or eliminate covalent bonding entirely.

Covalent bonding of (e.g. fiber to polymer) results from the overlap of electron clouds surrounding atoms forming a direct covalent bond between atomic centers. The covalent bond strengths are substantial, are roughly equivalent to ionic bonding and tend to have somewhat smaller interatomic distances and are to be avoided.

The varied types of van der Waals' (VDW) forces in the polymer/fiber interaction are different than covalent and ionic bonding. These van der Waals' forces tend to be forces between molecules, not between atomic centers. In the composites of the claimed materials strong covalent or ionic bonding is avoided. Reactive coupling agents that bond polymer to fiber are not used. The fiber polymer composite as shown in the embodiments is formed with van der Waals bonding as modified by the IM coating.

The van der Waals' force existing between substantially non-polar uncharged molecules occurs in non-polar molecules, the force arises from the movement of electrons within the molecule. Because of the rapidity of motion within the electron cloud, the non-polar molecule attains a small but meaningful instantaneous charge as electron movement causes a temporary change in the polarization of the molecule. These minor fluctuations in charge result in the dispersion portion of the van der Waals' force.

Such VDW forces, because of the nature of the fluctuating polarization of the molecule, tend to be low in bond strength, typically 50 kJ mol⁻¹ or less. Further, the range at which the force becomes attractive is also substantially greater than ionic or covalent bonding and tends to be about 3-10 Å.

In the IM modified (van der Waals bonded) composite materials, we have found that the unique combination of fiber, the varying, but controlled, size and aspect ratio of the fiber component, the modification of the interaction between the fiber and the polymer result in the creation of a unique van der Waals' bonding. The minimal van der Waals' forces arise between molecules/aggregates/crystals and are created by the combination of fiber size, polymer and interfacial modifiers in the composite.

In the past, materials have been made as mere mixtures of components or as covalently coupled components. While these are often characterized as “composite”, they are stiff inextensible materials or merely comprise a polymer filled with particulate with little or no van der Waals' interaction between the particulate filler material. In sharp contrast to the previous materials, the interaction between the selection of fiber size distribution and interfacially modified fiber enables the fiber to achieve an intermolecular distance that creates a certain level of van der Waals' bonding.

The coupled or crosslinked materials having little viscoelastic properties, do not achieve a true composite structure as now described. This leads us to conclude that this VDW intermolecular bonds or distance is not attained in the reference materials.

The fibers are typically coated with an interfacial surface chemical treatment also called an interfacial modifier (IM) that supports or enhances the final properties of the composite such as viscoelasticity, rheology, high packing fraction, and fiber surface inertness. These properties are not present in contemporary composite materials. The fibers can be coated separately, or the fibers can be combined and then coated in batch with the interfacial modifier. An interfacially modified fiber has a substantially complete, uniform coating over the surface, the interfacial modifier (IM) with a thickness of less than 1500 Angstroms often less than 200 Angstroms, and commonly 10 to 500 Angstroms (Å) or 100 to 1500 Angstroms (Å).

An interfacial modifier can be an organo-metallic material that provides an exterior coating on the fiber promoting the close association, but not attachment or bonding, of either polymer to fiber and fiber to fiber. The composite properties, such as fiber surface inertness, high volume packing of the coated fiber, rheology of the composite material, and viscoelastic properties of the composite, arise from the intimate association (VDW) of the polymer and fiber obtained by use of careful processing and manufacture. An interfacial modifier is an organic material, in some examples an organo-metallic material, that provides an exterior coating on the fiber to provide a surface that can associate with the polymer promoting the close association of polymer and fiber but with no reactive bonding, such as covalent bonding for example, of polymer to fiber, fiber to fiber, or fiber to a different particulate, such as a glass fiber or a glass bubble. The lack of reactive bonding between the components of the composite leads to the formation of the novel composite—such as high packing fraction, commercially useful rheology, viscoelastic properties, and surface inertness of the fiber. These characteristics can be readily observed when the composite with interfacially modified coated fiber is compared to fiber lacking the interfacial modifier coating or to fiber that is reactively coupled to other fiber or polymer. In one embodiment, the coating of interfacial modifier at least partially covers the surface of the fiber. In another embodiment, the coating of interfacial modifier continuously and uniformly covers the surface of the fiber, in a continuous coating phase layer.

Interfacial modifiers used in the application fall into broad categories including, for example, titanate compounds, zirconate compounds, hafnium compounds, samarium compounds, strontium compounds, neodymium compounds, yttrium compounds, phosphonate compounds, aluminate compounds and zinc compounds. Aluminates, phosphonates, titanates and zirconates that are useful contain from about 1 to about 3 ligands comprising hydrocarbyl phosphate esters and/or hydrocarbyl sulfonate esters and about 1 to 3 hydrocarbyl ligands which may further contain unsaturation and heteroatoms such as oxygen, nitrogen and sulfur.

In one embodiment, the interfacial modifier that can be used is a type of organo-metallic material such as organo-cobalt, organo-iron, organo-nickel, organo-titanate, organo-aluminate organo-strontium, organo-neodymium, organo-yttrium, organo-zinc or organo-zirconate. The specific type of organo-titanate, organo-aluminates, organo-strontium, organo-neodymium, organo-yttrium, organo-zirconates which can be used and which can be referred to as organo-metallic compounds are distinguished by the presence of at least one hydrolysable group and at least one organic moiety. Mixtures of the organo-metallic materials may be used. The mixture of the interfacial modifiers may be applied inter- or intra-fiber, which means at least one fiber may have more than one interfacial modifier coating the surface (intra), or more than one interfacial modifier coating may be applied to different fibers or fiber size distributions (inter).

Certain of these types of compounds may be defined by the following general formula:

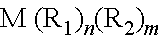

wherein M is a central atom selected from such metals as, for example, Ti, Al, and Zr and other metal centers; R₁ is a hydrolysable group; R₂ is a group consisting of an organic moiety, preferably an organic group that is non-reactive with polymer or other film former; wherein the sum of m+n must equal the coordination number of the central atom and where n is an integer ≥1 and m is an integer ≥1. Particularly R₁ is an alkoxy group having less than 12 carbon atoms. Other useful groups are those alkoxy groups, which have less than 6 carbons, and alkoxy groups having 1-3 C atoms. R₂ is an organic group including between 6-30, preferably 10-24 carbon atoms optionally including one or more hetero atoms selected from the group consisting of N, O, S and P. R₂ is a group consisting of an organic moiety, which is not easily hydrolyzed and is often lipophilic and can be a chain of an alkyl, ether, ester, organo-alkyl, organo-alkyl, organo-lipid, or organo-amine. The phosphorus may be present as phosphate, or phosphite groups. Furthermore, R₂ may be linear, branched, cyclic, or aromatic. R₂ is substantially unreactive, i.e. not providing attachment or bonding, to other particles or fiber within the composite material. Titanates provide antioxidant properties and can modify or control cure chemistry

The use of an interfacial modifier results in workable thermoplastic viscosity and improved structural properties in a final use such as a structural member or shaped article. Minimal amounts of the modifier can be used including about 0.005 to 8 wt.-%, about 0.01 to 6 wt.-%, about 0.02 to 5 wt.-%, or about 0.02 to 3 wt. %. The IM coating can be formed as a coating of at least 3 molecular layers or at least about 50 or about 100 to 500 or about 100 to 1000 or about 100 to 1500 angstroms (Å).

The claimed composites with increased loadings of fiber can be safely compounded and melt processed formed into high strength structural members. The interfacial modification technology depends on the ability to isolate the fibers within the continuous polymer phase. The isolation is obtained from a continuous molecular layer(s) of interfacial modifier to be distributed over the fiber surface. From another perspective, the IM coated fiber is immiscible in the polymer phase. Once this layer is applied, the behavior at the interface of the interfacial modifier coating to polymer dominates and defines the physical properties of the composite and the shaped or structural article (e.g. modulus, tensile, rheology, packing fraction and elongation behavior) while the bulk nature of the fiber dominates the bulk material characteristics of the composite (e.g. density, thermal conductivity, compressive strength). The correlation of fiber bulk properties to that of the final composite is especially strong due to the high-volume percentage loadings of discontinuous phase, such as fiber, associated with the technology.

The fibers are coated with IM to obtain the processing and physical properties needed. Once coated, the fiber exterior appears to be the IM composition to the polymer while the fiber character is hidden. The organic nature of the IM coating changes the nature of the interaction between the fiber surface and the polymer phase. The silicate surfaces of the fibers are of a different surface energy and hydrophobicity than the polymer or coating. The polymer does not easily associate with the inorganic fiber surface, but much more easily associates with the organic nature of the IM coated inorganic fiber surface. The IM coated fiber mixes well with the polymer and can achieve greater composite uniformity and fiber loadings.

The benefit of interfacial modification on a fully coated fiber is independent of overall fiber shape. The current upper limit constraint is associated with challenges of successful dispersion of fibers within laboratory compounding equipment without significantly damaging the high aspect ratio fibers. Furthermore, inherent rheological challenges are associated with high aspect ratio fibers. With proper engineering, the ability to successfully compound and produce interfacially modified fibers of fiber fragments with aspect ratio more than 20 often in excess of 100, 200 or more is provided.

The composite, thus, obtains improved physical properties such as heat deflection temperature (HDT) of greater than 60° C. (ASTM D648), IZOD impact strength of at least about 0.1, 0.4 or 1 ft-lb-in⁻¹ (0.2, 0.5 or 1.4 J)(ASTM D256), a COTE of less than about 5×10⁻⁵ or 1×10⁻⁶ cm·cm⁻-° C. (2×10⁻⁶ or 5×10⁻⁵ in/in·° F.) (ASTM 696), a tensile modulus (ASTM D638) of greater than about 0.7×10⁶ or 1×10⁶ psi at 72° F. (greater than about 4 or 5 kPa at 22° C.), a flexural modulus (ASTM D790) of greater than about 0.7×10⁶ or 1×10⁶ at 72° F. (greater than about 4 or 5 kPa 72° F.), a flexural strength (ASTM D790) of greater than 2.5×10³ psi or 5×10³ psi at 72° F. (5 mPa or 10 mPa at 22° C.), a tensile strength (ASTM D638) greater than about 2×10³ or 1×10⁴ psi at 72° F. (greater than about 4 or 1.5 kPa at 22° C.). Useful volume % of the fiber phase in the composite can be adjusted to above 40, 50, 60, 70, 80, or 90%, depending on the end use of the article or structural member and the required physical properties of the article or structural member, without loss of processability, viscoelasticity, rheology, high packing fraction, and fiber surface inertness of the composite.

A large variety of thermoplastic polymer and copolymer materials can be used in the composite materials. We have found that polymer materials useful in the composite include both condensation polymeric materials and addition or vinyl polymeric materials.

Vinyl polymers are typically manufactured by the polymerization of monomers having an ethylenically unsaturated olefinic group. Condensation polymers are typically prepared by a condensation polymerization reaction which is typically considered to be a stepwise chemical reaction in which two or more molecules combined, often but not necessarily accompanied by the separation of water, methanol or some other simple, typically volatile substance. Such polymers can be formed in a process called polycondensation. The typical polyvinyl chloride (PVC) polymer has a molecular weight (K) of at least about 50 or 60. Other polymers can be selected to match the physical properties of the PVC. Such polymers can have a density of at least 0.85 gm-cm⁻³, however, polymers having a density of greater than 0.96 are useful to enhance overall product density. A density is often 0.85 to 1.7 or up to 2 gm-cm⁻³ or can be about 0.96 to 1.95 gm-cm⁻³.

Vinyl polymers include polyacrylonitrile; polymer of alpha-olefins such as ethylene, propylene, etc.; polymers of chlorinated monomers such as vinyl chloride, vinylidene chloride, acrylate monomers such as acrylic acid, methyl acrylate, methyl methacrylate, acrylamide, hydroxyethyl acrylate, and others; styrenic monomers such as styrene, alpha-methyl styrene, vinyl toluene, etc.; vinyl acetate; and other commonly available ethylenically unsaturated monomer compositions. Examples include polyethylene, polypropylene, polybutylene, acrylonitrile-butadiene-styrene (ABS), polybutylene copolymers, polyacetal resins, polyactylic resins, homopolymers, etc.

Useful polymers are halogen polymers such as homopolymers, copolymers, and blends comprising vinyl chloride, vinylidene chloride, fluorocarbon monomers, etc. Polyvinyl chloride polymers with a K value of 50-75 can be used. A characteristic of the PVC resin is the length or size of the polymer molecules. A measure of the length or size is molecular weight of PVC. A useful molecular weight can be based on measurements of viscosity of a PVC solution. Such a K value ranges usually between 35 and 80. Low K-values imply low molecular weight (which is easy to process but has properties consistent with lower polymer size) and high K-values imply high molecular weight, (which is difficult to process, but has properties consistent with polymer size). The most commonly employed molecular characterization of PVC is to measure the one-point-solution viscosity. Expressed either as inherent viscosity (IV) or K-value, this measurement is used to select resins for the use in extrusion, molding, as well as for sheets, films or other applications. The inherent viscosity (IV) or K-value is the industry standard (ISO 1628-2) starting point for designing compounds for end use. Polymer solution viscosity is the most common measurement for further calculation of inherent viscosity or the K-value, because it is an inexpensive and routine procedure that can be used in manufacturing as well as in R&D labs. For example, a Lovis® 2000 M/ME micro-viscometer can measure polymer solution viscosity and set K value. A useful PVC is a blend of PVC homopolymer and 1-3 wt. % stabilizers, 1-4 wt. 5 process aids, 1-3 wt. % metal release aids, 1-5 wt. % internal and external lubricants, and up to 15 wt. % inorganic fillers.

Condensation polymers include nylon, phenoxy resins, polyarylether such as polyphenylether, polyphenylsulfide materials; polycarbonate materials, chlorinated polyether resins, polyethersulfone resins, polyphenylene oxide resins, polysulfone resins, polyimide resins, thermoplastic urethane elastomers and many other resin materials. Condensation polymers that can be used in the composite materials include polyamides, polyamide-imide polymers, polyarylsulfones, polycarbonate, polybutylene terephthalate, polybutylene naphthalate, polyetherimides, polyether sulfones, polyethylene terephthalate, thermoplastic polyamides, polyphenylene ether blends, polyphenylene sulfide, polysulfones, thermoplastic polyurethanes and others. Preferred condensation engineering polymers include polycarbonate materials, polyphenyleneoxide materials, and polyester materials including polyethylene terephthalate, polybutylene terephthalate, polyethylene naphthalate and polybutylene naphthalate materials. For composites containing high volumetric loading of fibers, the rheological behavior of the highly packed composites depends on the characteristics of the contact points between the fibers and the distance between fibers. When forming composites with polymeric volumes approximately equal to the excluded volume of the discontinuous phase, inter-fiber interaction dominates the behavior of the material. Fibers contact one another and the combination of interacting sharp edges, soft surfaces (resulting in gouging) and the friction between the surfaces prevent further or optimal packing.

The surface of the interfacially modified fiber behaves as a fiber formed of the non-reacted end or non-reacting end of the interfacial modifier. This non-reacting portion of the interfacial modifier makes the fiber surface inert in reactivity. The coating of the interfacial modifier improves fiber surface wetting by the polymer and as a result improves the physical association of the fiber and polymer in the formed composite material leading to improved physical properties including, but not limited to, increased tensile and flexural strength, increased tensile and flexural modulus, improved notched IZOD or Gardner impact results and reduced coefficient of thermal expansion. In the melt, the interfacial modified coating on the fiber reduces the friction between fibers thereby preventing gouging and allowing for greater freedom of movement between fibers in contrast to fibers that have not been coated with interfacial modifier chemistry. Thus, the composite can be melt-processed at greater productivity and at conditions of reduced temperature and pressure severity. The process and physical property benefits of utilizing the coated fibers in the acceptable fiber morphology index range does not become evident until packing to a significant proportion of the maximum packing fraction; this value is typically about or greater than approximately 40, 50, 60, 70, 80, 90, 92, or 95 volume or weight % of the fiber phase in the composite.

In a composite, the fiber is usually much stronger and stiffer than the polymer matrix and gives the composite its designed structural or shaped article properties. The matrix holds the fiber in an orderly high-density pattern, Because the fibers are usually discontinuous, the matrix also helps to transfer load among the non-metal, inorganic, synthetic, natural, or mineral fibers such as the calcium silicates. Processing can aid in the mixing and filling of the non-metal, inorganic or mineral fibers. To aid in the mixture, an interfacial modifier can help to overcome the forces that prevent the polymer matrix from forming a substantially continuous phase of the composite. The tunable composite properties arise from the intimate association of the fiber and the polymer obtained by the use of careful polymer processing and manufacture. We believe an interfacial modifier (IM) is an organic material that provides an exterior coating on the fiber promoting the close association of polymer and fiber. Minimal amounts of the interfacial modifier can be used on regular morphology while higher amounts of the IM are used to coat materials with increased or irregular surface morphology. Typically, the composite materials can be manufactured using melt processing and are also utilized in product formation using melt processing such as extrusion, injection molding and the like.

Typically, the composite materials can be manufactured using melt processing and are also utilized in product formation using melt processing. A typical thermoplastic polymer material is combined with IM coated fiber and processed until the material attains a uniform density (if density is the characteristic used as a determinant). Once the material attains a sufficient property, such as, for example, density, the material can be extruded into a product or into an intermediate article in the form of a pellet, chip, wafer, preform or other easily processed material using conventional processing techniques.

In the manufacture of useful products, the manufactured composite can be obtained in appropriate amounts, subjected to heat and pressure, typically in an extruder, or in additive manufacturing useful for 3D printing (additive manufacturing), or injection molding equipment and then formed into an appropriate shape having the correct amount of composite materials in the appropriate physical configuration.

In the appropriate product design, during composite manufacture or during product or article manufacture, a pigment or other dye material can be added to the processing equipment. One advantage of this material is that an inorganic dye or pigment can be co-processed resulting in a material that needs no exterior painting or coating to obtain an attractive, functional, or decorative appearance. The pigments can be included in the polymer blend, can be uniformly distributed throughout the material and can result in a surface that cannot chip, scar or lose its decorative appearance. One particularly important pigment material comprises titanium dioxide (TiO₂). This material is non-toxic, is a bright white particulate that can be easily combined with the fiber and/or polymer composites to enhance the novel characteristics of the composite material and to provide a white hue to the ultimate composite material.

The manufacture of the composite materials depends on good melt processing manufacturing technique. The fiber is initially treated with an interfacial modifier by contacting the fiber surface with the modifier in the form of a solution of interfacial modifier on the fiber with blending and drying carefully to ensure a uniform IM coating over the fiber surface. Interfacial modifier can also be added to fibers in bulk blending operations using high intensity Littleford or Henschel blenders. Alternatively, addition of the fiber to the twin cone mixers can be followed by drying or direct addition to a screw compounding device. Interfacial modifiers may also be combined with the fiber or particulate in aprotic solvent such as toluene, tetrahydrofuran, mineral spirits or other such known solvents.

The fiber can be combined into the polymer phase depending on the nature of the polymer phase the fiber surface chemistry and any pigment process aid or additive present in the composite material. The composite materials having the desired physical properties can be manufactured as follows. In an embodiment, the surface of the fiber is initially prepared, the interfacial modifier coats the fiber, and the resulting product is isolated and then combined with the continuous polymer phase to affect an immiscible dispersion or association between the IM coated fiber and the polymer. Once the composite material is compounded or prepared, it is then melt-processed into the desired shape of the end use article.

Solution processing is an alternative that provides solvent recovery during materials processing. The materials can also be dry blended without solvent. Blending systems such as ribbon blenders obtained from Drais Systems, high-density drive blenders available from Littleford Brothers and Henschel are possible. Further melt blending using Banberry, other single screw or twin-screw compounders is also useful. When the materials are processed as a plastisol or organosols with solvent, liquid ingredients are generally charged to a processing unit first, followed by polymer, fiber and rapid agitation. Once all materials are added a vacuum can be applied to remove residual air and solvent, and mixing is continued until the product is uniform and high in density.

Dry blending is generally preferred due to advantages in cost. However certain embodiments can be compositionally unstable due to differences in fiber size. In dry blending processes, the composite can be made by first introducing the polymer, combining the polymer stabilizers, if necessary, at a temperature from about ambient to about 60° C. with the polymer, blending an IM coated fiber with the stabilized polymer, blending other process aids, colorants, indicators or lubricants followed by mixing in hot mix, transfer to storage, packaging or end use manufacture. Interfacially modified materials can be made with solvent techniques that use an effective amount of solvent to initiate formation of a composite.

The improved process viscosity can be seen in comparing the processing of a composite as claimed compared to a composite of uncoated fiber. The claimed materials have substantially reduced processing viscosity that is derived from the freedom of movement of the interfacially modified fiber within the polymer matrix. An IM also provides some fiber self-ordering within the polymer matrix which increases the fiber packing fraction without the loss of rheology or breakage of fibers.

Interfacially modified materials can be made with solvent techniques that use an effective amount of solvent to initiate formation of a composite. When interfacially modification is substantially complete, the solvent can be stripped. Such solvent processes are conducted by solvating the interfacial modifier or polymer or both; mixing a CaSiO₃ fiber, such as wollastonite, with interfacial modifier into a bulk phase or polymer master batch and devolatilizing the composition in the presence of heat & vacuum above the Tg of the polymer.

When compounding with twin screw compounders or extruders, a process can be used employing twin screw compounding can be described as adding the CaSiO₃ fiber and raise temperature to remove surface water (barrel 1); adding interfacial modifier to the twin screw when the CaSiO₃ fiber is at temperature (barrel 3); dispersing or distributing interfacial modifier on the CaSiO₃ fiber; maintaining reaction temperature to completion; venting reaction by-products (barrel 6), adding polymer (barrel 7); compressing and/or melting polymer; dispersing or distributing polymer in the CaSiO₃ fiber; reacting interfacially modified CaSiO₃ fiber with polymer binder; degassing remaining reaction products (barrel 9), compressing resulting CaSiO₃ fiber and polymer composite and forming the desired shape, pellet, lineal, tube, injection mold article, etc. through a die or post-manufacturing step.

In another embodiment for formulations of pre-sized CaSiO₃ fibers, a process could be adding polymer; raising the temperature of the polymer to a melt state; adding CaSiO₃ fiber which has been pre-treated with the interfacial modifier; dispersing and distributing the IM pre-treated CaSiO₃ fiber in the polymer; compressing the resulting CaSiO₃ fiber and polymer composite; and forming the desired shape, pellet, lineal, tube, injection mold article, etc. through a die or post-manufacturing step.

The composite can be used to make an article of manufacture. Such articles can be made directly from the compounding process or can be made from a pellet input. Articles can include pellets used in further thermoplastic processing, structural members, or other articles that can be made using thermoplastic processing such as injection molding, compression molding, etc. The pellet is a roughly cylindrical, ovoid or egg-shaped or object that can be fed into an extruder input. The pellet is typically 1 to 50 mm, 1 to 40 mm, 1 to 30 mm, or 1 to 20 mm in length and 1 to 3 mm, 1 to 6 mm, or 1 to 8 mm or 1 to 10 mm in diameter and the length to diameter ratio, L:D, is less than 5 or 2. The ovoid pellet is typically 1 to 40 mm in length and 1 to 20 mm in diameter. A pellet weighs about 10 to 100 mg, 10 to 80 mg, 10 to 70 mg, 10 to 60 mg, 10 to 50 mg, 20 to 50 mg, 20 to 60 mg, 20 to 70 mg, 20 to 80 mg.

Structural members include linear extrudate that can be mechanically milled or reinforced with secondary members. The articles can be used in a fenestration unit as a frame member, muntin, grill etc. The articles can be used in a decking installation as a decking member, a trim, or a support. The article can be used as a rail, baluster or post. The article can be used as a siding member.

The interior of the structural member is commonly provided with one or more structural webs which in a direction of applied stress supports the structure. Structural web typically comprises a wall, post, support member, or other formed structural element which increases compressive strength, torsion strength, or other structural or mechanical properties. Such structural web connects the adjacent or opposing surfaces of the interior of the structural member. More than one structural web can be placed to carry stress from surface to surface at the locations of the application of stress to protect the structural member from crushing, torsional failure or general breakage. Typically, such support webs are extruded, or injection molded during the manufacture of the structural material. However, a support can be post added from parts made during separate manufacturing operations.

The internal space of the structural member can also contain a fastener anchor or fastener installation support. Such an anchor or support means provides a locus for the introduction of a screw, nail, bolt or other fastener used in either assembling the unit or anchoring the unit to a rough opening in the commercial or residential structure. The anchor web typically is conformed to adapt itself to the geometry of the anchor and can simply comprise an angular opening in a formed composite structure, can comprise opposing surfaces having a gap or valley approximately equal to the screw thickness, can be geometrically formed to match a key or other lock mechanism, or can take the form of any commonly available automatic fastener means available to the window manufacturer from fastener or anchor parts manufactured by companies such as Amerock Corp., Illinois Tool Works and others.

The structural member can have extrusion molded, premolded paths or paths machined into the molded thermoplastic composite for passage of door or window units, fasteners such as screws, nails, etc. Such paths can be counter sunk, metal lined, or otherwise adapted to the geometry or the composition of the fastener materials. The structural member can have mating surfaces premolded in order to provide rapid assembly with other components or another window. Components of similar or different compositions having similarly adapted mating surfaces. Further, the structural member can have mating surfaces formed in the shell of the structural member adapted to moveable window sash or door sash or other moveable parts used in window operations.

The structural member can have a mating surface adapted for the attachment of the subfloor or base, framing studs or side molding or beam, top portion of the structural member to the rough opening. Such a mating surface can be flat or can have a geometry designed to permit easy installation, sufficient support and attachment to the rough opening. The structural member shell can have other surfaces adapted to an exterior trim and interior mating with wood trim pieces and other surfaces formed into the exposed sides of the structural member adapted to the installation of metal runners, wood trim parts, door runner supports, or other metal, plastic, or wood members commonly used in the assembly of windows and doors.

The structural members can be assembled with a variety of known mechanical fastener techniques. Such techniques include screws, nails, and other hardware. The structural members can also be joined by an insert into the hollow profile, glue, or a melt fusing technique wherein a fused weld is formed at a joint between two structural members. The structural members can be cut or milled to form conventional mating surfaces including 90° angle joints, rabbit joints, tongue and groove joints, butt joints, etc. Such joints can be bonded using an insert placed into the hollow profile that is hidden when joinery is complete. Such an insert can be glued or thermally welded into place. The insert can be injection molded or formed from similar thermoplastics and can have a surface adapted for compression fitting and secure attachment to the structural member. Such an insert can project from approximately 1 to 5 inches into the hollow interior of the structural member. The insert can be shaped to form a 90° angle, a 1800 extension, or other acute or obtuse angle required in the assembly of the structural member. Further, such members can be manufactured by milling the mating faces and gluing members together with a solvent, structural or hot melt adhesive. Solvent borne adhesives that can act to dissolve or soften thermoplastic present in the structural member and to promote solvent based adhesion or welding of the materials are known in polyvinyl chloride technology. In the welding technique, once the joint surfaces are formed, the surfaces of the joint can be heated to a temperature above the melting point of the composite material and while hot, the mating surfaces can be contacted in a configuration required in the assembled structure. The contacted heated surfaces fuse through an intimate mixing of molten thermoplastic from each surface. Once mixed, the materials cool to form a structural joint having strength typically greater than joinery made with conventional techniques. Any excess thermoplastic melt that is forced from the joint area by pressure in assembling the surfaces can be removed.

TABLE 1
Exemplary Composites

| Component | Useful amounts | Useful amounts | Useful amounts |
|---|---|---|---|
| Silicate fiber | 5-65 | 10-60 | 10-55 |
| (vol. %) | | | |
| Silicate fiber | 30-80 | 35-75 | 40-70 |
| (wt. %) | | | |
| Interfacial | 0.1-5 | 0.3-3 | 0.5-2.5 |
| modifier coating | | | |
| (Vol. %) | | | |
| IM coating | 0.1-2 | 0.2-1 | 0.3-0.8 |
| (Wt. %) | | | |
| Thermoplastic | 35-95 | 40-90 | 45-90 |
| polymer | | | |
| (vol. %) | | | |
| Thermoplastic | 20-70 | 25-65 | 30-60 |
| polymer | | | |
| (wt. %) | | | |

TABLE 2
Typical property ranges
With an IM, the composites can achieve the following properties:

| | ASTM | Minimum | | Range of | |
|---|---|---|---|---|---|
| Property | Method | Value | | Value | Units |
| Tensile Strength | D638 | 35 | (5 × 10⁴) | >35 | MPa (psi) |
| Tensile Modulus | D638 | 8 | (1.2 × 10⁶) | >4 | GPa (psi) |
| Flexural Strength | D790 | 35 | (5 × 10¹) | >35 | MPa (psi) |
| Flexural Modulus | D790 | 8 | (1.2 × 10⁶) | >4 | GPa (psi) |
| Notched IZOD | D256 | 70 | (1.3) | >30 | J · m⁻¹ |
| | | | | | (ft · lbs · in⁻¹) |
| HDT | D648 | Greater than 60 | | 65-95 | º C. |
| COTE | D969 | Maximum Value: | | <5.4 × 10⁻⁵ | cm · cm⁻³ · º C. |
| | | 1.9 × 10⁻⁵ | (1.1 × 10⁻⁵) | | (in · in⁻¹ · º F.) |

Additionally, the use of the IM permits an increase in the fiber loading in the composite. As the fiber content increases, the polymer content decreases. The thermal expansion of a structural member made with the disclosed material will be improved as fiber content increases, e.g., the material will have reduced coefficient of thermal expansion (COTE).

### EXPERIMENTAL SECTION

In a 70-mm compounder, a pellet was extruded at 168-171° C. using conventional compounding rates and then extruded into test piece(s) for physical testing as shown below using the proportions as follows and in Table 3:

### Example 3

Polyvinyl chloride homopolymer (PVC), Formolon ASW16 (Formosa, Livingston, NJ) was combined with an IM coated fiber (organo titanate, 1 wt. %) wollastonite fiber, (Vansil VG) to form a concentrate Master batch. This was compounded into a pellet with dimensions of approximate length of 3 mm and approximate diameter of 5 mm.

### Example 1

The master batch was combined with PVC to form a test piece containing 4 vol. % (7 wt. %) wollastonite for tensile testing.

### Example 2

The master batch was combined with PVC to form a test piece containing 23 vol. % (37 wt. %) wollastonite for physical properties testing.

### Compounder Configuration:

| | | |
|---|---|---|
| Barrel Temperature | 191 | ° C. |
| rate | 6-7 | RPM |
| Throughput | 1500 | ft-min⁻¹ |
| Die | Pellet die | |
| Pelletizer speed | 700 | RPM |
| Vacuum | 20 | In − Hg |

The compounded pellets were extruded into test strips on a 1″ diameter extruder. Extruded strips were cut to length and tested for tensile properties (ASTM D638), Izod impact (ASTM D256), heat deflection temperature (HDT) (ASTM D648), and coefficient of thermal expansion (ASTM D696).

TABLE 3
Compositions

| Material | Wt. % | Vol. % | |
|---|---|---|---|
| Example | Wollastonite | Wollastonite | |
| No. | Fiber | Fiber | g-cm⁻³ |
| 1 | 7% | 4% | 1.48 |
| 2 | 37% | 23% | 1.76 |
| 3 | 70% | 53% | 2.22 |

TABLE 4
Physical testing results

| | | | | | IZOD | | |
|---|---|---|---|---|---|---|---|
| | Tensile | | Flex | | Impact | | |
| | Testing | | Testing | | Strength | Thermal | |
| | Max | | Max | | Notched | Properties | |
| | Tensile | Tensile | Flexural | Flexural | Impact | | HDT |
| | Strength | Modulus | Strength | Modulus | Strength | COTE | (264 PSI) |
| | Parameter | | | | | | |
| | psi | Mpsi | psi | Mpsi | ft-lbf-in⁻² | in/in ° C. | ° C. |
| Material | ASTM# | | | | | | |
| Example No. | D638 | D638 | D790 | D790 | D256 | D696 | D648 |
| 1 | 4796 | 0.50 | 8532 | 0.42 | 1.5 | 4.97E−05 | 66.4 |
| 2 | 4011 | 1.17 | 8256 | 1.35 | 2.4 | 2.44E−05 | 68.6 |
| 3 | 2600 | 2.12 | 4800 | 2.49 | 1.67 | 4.00−06 | 65.9 |

### Example 4

Polyvinyl chloride homopolymer, Formolon® AWS16 (Formosa, Livingston, NJ) in an amount of 69 vol. % (53 wt. %) was combined with an IM coated fiber (organo titanate, 0.88 vol. %, 0.46 wt. %) wollastonite fiber, length 65 μm and diameter 7 μm (HR2000, Vansil) in an amount of 30 vol. % (46 wt. %) and compounded into a pellet with dimensions of approximate length of 3 mm and approximate diameter of 5 mm. Comparative examples were made with glass fiber components.

### Comparative Example 4a

Polyvinyl chloride homopolymer, Formolon® AWS16 (Formosa, Livingston, NJ) in an amount of 68 vol. % (54 wt. %) combined with an IM coated fiber (organotitanate, 1.11 vol. %, 0.64 wt. %) of glass fiber (John Mansville, Starstran 718) in an amount of 29 vol. % (43 wt. %) and CaCO₃ in an amount of 1 vol. % (2 wt. %) and compounded into a pellet with dimensions of approximate length of 3 mm and approximate diameter of 5 mm.

### Comparative Example 4b

Polyvinyl chloride homopolymer, Formolon® AWS16 (Formosa, Livingston, NJ) in an amount of 58 vol. % (44 wt. %) combined with an IM coated fiber (organotitanate, 1.69 vol. %, 0.83 wt. %), glass fiber (John Mansville, Starstran 718) in an amount of 41 vol. % (55 wt. %) and compounded into a pellet with dimensions of approximate length of 3 mm and approximate diameter of 5 mm.

### Comparative Example 4c

Polyvinyl chloride homopolymer, Formolon® AWS16 (Formosa, Livingston, NJ) in an amount of 58 vol. % (44 wt. %) combined with an IM coated fiber (organotitanate, 1.69 vol. %, 0.83 wt. %), glass bead (John Mansville, Starstran 718) in an amount of 41 vol. % (55 wt. %) and compounded into a pellet with dimensions of approximate length of 3 mm and approximate diameter of 5 mm. The Example and the comparative examples were tested using the appropriate test piece as follows in Table 5.

TABLE 5
testing results

| Property | Example 4 | Comp. Ex. 4a | Comp. Ex. 4b |
|---|---|---|---|
| Density (g-cc⁻¹) | 1.87 | 1.80 | 1.87 |
| Tensile strength (psi) | 3383 | 7413 | 9000 |
| Tensile modulus (Mpsi) | 1.24 | 1.67 | 2.06 |
| Tensile elongation (%) | 1.4 | 0.75 | 1.0 |
| Flex strength (psi) | 7726 | 13285 | 16900 |
| Flex modulus (Mpsi) | 1.33 | 1.97 | 2.48 |
| Heat distortion temp. (° C.) | 69 | 74 | 75 |
| COTE (in · in⁻¹ · º F.) | 1.4 × 10⁻⁵ | 1.1 × 10⁻⁵ | 4.6 × 10⁻⁶ |
| Notched IZOD | 1.6 | 2.2 | 3.0 |
| impact strength (ft · lbs · in⁻²) | | | |
| Un-notched IZOD | 13 | 3.73 | |
| impact strength (ft · lbs · in⁻²) | | | |
| Supported drop | 14 (11) | 7 (18) | 18 |
| dart energy (J) at height (in) | | | |
| Unsupported drop | 9.4 (11) | 5.2 (11.4) | |
| dart energy (J) | | | |

TABLE 6
Examples 5-8 - Comparative and Test Results
Following the procedures set forth for Examples 1-3 the following materials were made and tested.

| | | | Example | | | | |
|---|---|---|---|---|---|---|---|
| | | | Ex. 5 | Ex. 6 | | Ex. 7 | Ex. 8 |
| | | | Wollastonite | Wollastonite | Comp Ex. 4c | Wollastonite | Wollastonite |
| | | | Vansil | Vansil | Glass | Vansil | Vansil |
| | | | HR1500 | HR1500 | Bead | HR2400 | HR2400 |
| Particle Fill | | vol % | 30% | 45% | 29% | 29% | 43% |
| Puck | Puck Density | g/cc | | | 1.760 | 1.860 | 2.030 |
| Bulk | Bulk Density | g/cc | | | 0.08 | | |
| Extrusion | Melt Pressure | psi | 2170 | 3800 | 1900 | 2280 | 2870 |
| | Melt Temperature | ° C. | 167 | 169 | 173 | 168 | 169 |
| Tensile | Tensile Strength | psi | 3542 | 2299 | 7413 | 3383 | 2634 |
| | Tensile Modulus | Mpsi | 1.41 | 1.47 | 1.67 | 1.241 | 1.502 |
| | Tensile Elongation | % | 1.44 | 0.53 | 0.75 | 1.71 | 0.79 |
| Flex | Flex | psi | 6975 | 4376 | 13285 | 7726 | 5836 |
| | Flex Modulus | Mpsi | 1.28 | 1.44 | 1.97 | 1.332 | 2.026 |
| | Flex Toughness | lb · ft · in · in⁻³ | 130 | 29 | 62 | 179.4 | 50.87 |
| HDT | HDT | ° C. | 69.2 | 68.9 | 73.6 | 70.1 | 70.9 |
| COTE | COTE | in-in · ° F.⁻¹ | | | 1.11E−05 | 1.4E−05 | 8.66E−06 |
| IZOD | Un-Notched IZOD | ft · lb · in⁻¹ | 4.5 | 1.73 | 1.82 | 7.33 | 2.95 |
| | Impact Resistance | | | | | | |
| | Un-Notched IZOD | ft · lb · in⁻² | 9 | 3 | 4 | 13 | 5.87 |
| | Impact Strength | | | | | | |
| Notched IZOD | Notched IZOD | ft · lb · in⁻² | 0.56 | 0.570 | 0.800 | 0.880 | 0.660 |
| | Impact Resistance | | | | | | |
| Notched IZOD | Notched IZOD | ft · lb · in⁻² | 1.61 | 1.58 | 2.06 | 2.04 | 1.57 |
| | impact strength | | | | | | |
| Un-Supported | Un-Supported Drop | Joules | 12.75 | 14.74 | 10.36 | 9.86 | 10.64 |
| Drop Dart | Dart Energy | | | | | | |
| Supported Drop | Supported Drop Dart | Joules | 1.8.64 | 17.22 | 10.02 | 16.03 | 17.53 |
| Dart | Energy | | | | | | |

### Example 9

Wollastonite was first coated with an organo titanate interfacial modifier (1 wt. %) using a coater process. The coated wollastonite was then compounded into a composite with PVC (either PolyOne®, Fiberloc® or Formosa® AWS160 on a 26 mm compounder. A range of materials were made containing 70 to 40 wt. % PVC and 30 to 60 wt. % wollastonite. Compounded pellets were made of the materials.

TABLE 8
Compounder configuration:

| | | |
|---|---|---|
| Barrel Temperature | 170-190 | ° C. |
| Screw Speed | 112 | RPM |
| Throughput | 38 | lb-hr-1 |
| Die | Pellet die | |
| | 6-hole × 3 mm | |
| Pelletizer speed | 700 | RPM |
| Vacuum | 20 | In − Hg |

TABLE 9
Extruder configuration:
The compounded pellets were extruded
into test strips on a 1″ diameter extruder
under the following configuration:

| | Standard auger | |
|---|---|---|
| Screw Design | (1:1 compression ratio) | |
| Screw Speed | 25 | RPM |
| Barrel Temperature | 180-190 | ° C. |
| Die Temperature | 205 | ° C. |

Extruded strips were cut to length and tested for tensile properties (ASTM D638), Izod impact (ASTM D256), heat deflection temperature (HDT) (ASTM D648), and coefficient of thermal expansion (ASTM D696).

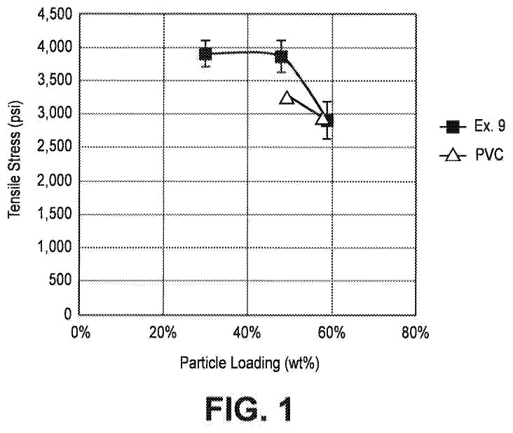

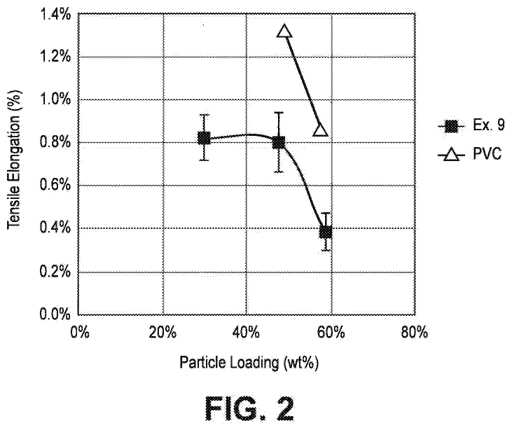

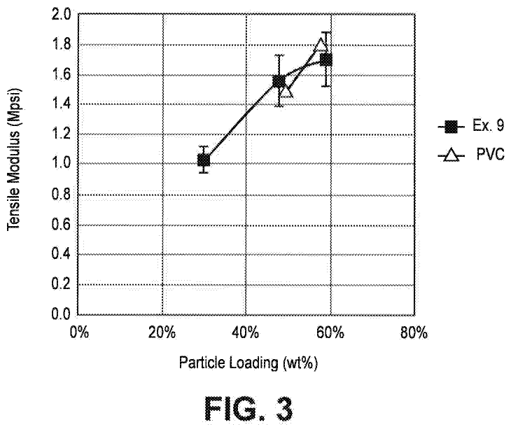

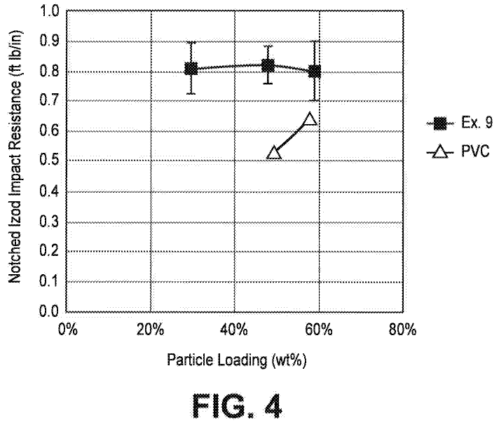

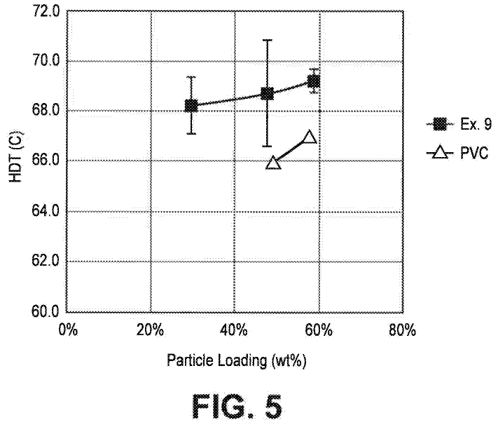

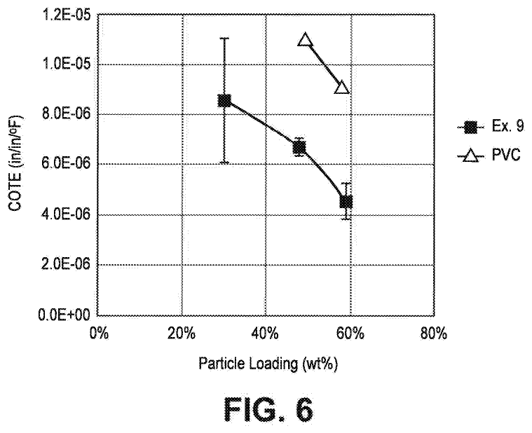

FIGS. 1-6 Show the results of testing Example 9 for its physical properties including tensile strength, tensile elongation, tensile modulus, notched Izod impact resistance, heat deflection temperature, and coefficient of thermal expansion. These data show that wollastonite improves the tensile, impact and heat resistance properties of PVC.

Following the procedures set forth for Examples 1-3 the following materials were made and tested.

TABLE 10
Examples 10-12 - Compared to Examples 1-3 and Test Results

| | | Wt. % | Vol. % | Wt. % | vol % | | | |
|---|---|---|---|---|---|---|---|---|
| Material | Pine | wollastonite | wollastonite | Pine | Pine | Wt. % | Vol. % | |
| Example no. | fiber | Fiber | Fiber | Fiber | Fiber | solids | solids | g/cc |
| 1 | None | 7% | 4% | 0% | 0% | 7% | 4% | 1.48 |
| 2 | None | 37% | 23% | 0% | 0% | 37% | 23% | 1.76 |
| 3 | None | 70% | 53% | 0% | 0% | 70% | 54% | 2.22 |
| 10 | Pine | 30% | 17% | 10% | 11% | 40% | 30% | 1.66 |
| | Fiber | | | | | | | |
| 11 | Pine | 10% | 5% | 30% | 30% | 40% | 38% | 1.48 |
| | Fiber | | | | | | | |
| 12 | Pine | 45% | 28% | 15% | 18% | 60% | 49% | 1.83 |
| | Fiber | | | | | | | |

TABLE 11
Test results

| | Tensile | | Flex | | IZOD Impact | Thermal | |
|---|---|---|---|---|---|---|---|
| | Testing | | Testing | | Strength | Properties | |
| | Test | | | | | | |
| | | | | | | | Heat |
| | Max | | Max | | Notched | | Deflection |
| | Tensile | Tensile | Flexural | Flexural | Impact | | Temperature |
| | Strength | Modulus | Strength | Modulus | Strength | COTE | (264 PSI) |
| | Parameter | | | | | | |
| | psi | Mpsi | psi | Mpsi | ft-lbf-in⁻² | in/in ° C. | ° C. |
| Material | ASTM # | | | | | | |
| Example No. | D638 | D638 | D790 | D790 | D256 | D696 | D648 |
| Example | 4796 | 0.50 | 8532 | 0.42 | 1.5 | 4.97E−05 | 66.4 |
| 1 | 4011 | 1.17 | 8256 | 1.35 | 2.4 | 2.44E−05 | 68.6 |
| 2 | 2600 | 2.12 | 4800 | 2.49 | 1.6 | 4.00E−06 | 65.9 |
| 3 | 3884 | 1.12 | 7719 | 1.13 | 3.2 | 2.24E−05 | 70.8 |
| 10 | 3503 | 0.80 | 6579 | 0.75 | 2.8 | 2.38E−05 | 71.3 |
| 11 | 2906 | 1.48 | 5133 | 1.02 | 2.3 | 1.90E−05 | 72.6 |
| 12 | | | | | | | |

These data support the conclusion that the interfacial modifier can improve the compatibility of an array of materials in the composite. These data support the conclusion that the wollastonite composite materials have excellent physical properties including tensile strength, tensile elongation, tensile modulus, notched Izod impact resistance, heat deflection temperature, and coefficient of thermal expansion. These materials show a cooperation with the glass bead and fiber and with organic fiber in improving engineering properties in structural members. FIGS. 1-6 Show the results of testing Example 9 versus standard PVC polymer formulations. These data show that wollastonite improves the tensile, impact and heat resistance properties of PVC polymer and other composites.

The claims may suitably comprise, consist of, or consist essentially of, or be substantially free or free of any of the disclosed or recited elements. The claimed technology is illustratively disclosed herein can also be suitably practiced in the absence of any element which is not specifically disclosed herein. The various embodiments described above are provided by way of illustration only and should not be construed to limit the claims attached hereto. Various modifications and changes may be made without following the example embodiments and applications illustrated and described herein, and without departing from the true spirit and scope of the following claims.

The specification shows an enabling disclosure of the composite technology, other embodiments may be made with the claimed materials. Accordingly, the invention is embodied solely in the claims hereinafter appended.

1-45. (canceled)

46. A thermoplastic composite comprising:
(a) about 30 to 55 wt. % of a wollastonite fiber, the fiber having a length of about 1 to 150 microns, a diameter greater than about 3 microns, an aspect ratio greater than about 3, the fiber having about 0.1 to 5 wt. % of an exterior coating comprising an organometallic interfacial modifier having a thickness of less than 1500 Å;
(b) about 10 to 20 wt. % of a glass fiber the glass fiber having about 0.1 to 5 wt. % of an exterior coating comprising an organometallic interfacial modifier having a thickness of less than 1500 Å; and
(c) the balance of the composite comprising a polymer phase comprising a vinyl chloride polymer
wherein the composite has a heat deflection temperature of at least 60° C. (ASTM D648), a notched IZOD impact resistance of about 0.4 to 3.0 ft-lb-in-1 (ASTM D256), a COTE of about less than 2×10-6 in/in-1/° F. (ASTM 696), a tensile modulus (ASTM D638) of greater than 700,000 psi at 72° F., a flexural modulus (ASTM D790) of greater than 700,000 psi at 72° F., a flexural strength (ASTM D790) of greater than 2,500 psi at 72° F., a tensile strength (ASTM D638) of greater than 2,000 psi at 72° F. and the weight percentages are based on the composite.

47. The composite of claim 46 wherein the composite comprises a vinyl chloride homopolymer.

48. The composite of claim 46 wherein the wollastonite fiber has a length about 50-250 microns, a diameter about 5-20 microns and an aspect ratio greater than about 15, the fiber having about 0.02 to 3 wt. % of the exterior coating, having a thickness of 10 to 500 Å, comprising an organometallic interfacial modifier.

49. The composite of claim 46 wherein the vinyl chloride polymer has a K value of about 50-75 (ISO 1628-2).

50. The composite of claim 49 wherein the wollastonite fiber has about 0.1 to 3 wt. % of an exterior coating comprising an organometallic interfacial modifier.

51. The composite of claim 49 wherein the glass fiber has about 0.1 to 3 wt. % of an exterior coating comprising an organometallic interfacial modifier.

52. The composite of claim 50 wherein the organometallic interfacial modifier comprises a titanate compound.

53. The composite of claim 51 wherein the organometallic interfacial modifier comprises a titanate compound.

54. An extrusion process forming a fiber polymer composite comprising the steps of:
(i) passing a PVC polymer through an extruder forming a melt stream;
(ii) combining the melt stream with a first fiber master batch forming a fiber stream; and
(iii) combining the fiber stream with a second fiber forming a uniform fiber composite;
wherein the composite comprises:
(a) about 30 to 55 wt. % of a wollastonite fiber, the fiber having a length of about 1 to 150 microns, a diameter greater than about 3 microns, an aspect ratio greater than about 3, the fiber having about 0.1 to 5 wt. % of an exterior coating comprising an organometallic interfacial modifier having a thickness of less than 1500 Å;
(b) about 10 to 20 wt. % of a glass fiber the glass fiber having about 0.1 to 5 wt. % of an exterior coating comprising an organometallic interfacial modifier having a thickness of less than 1500 Å; and
(c) the balance of the composite comprising a polymer phase comprising a vinyl chloride polymer.

55. The process of claim 54 wherein the first fiber master batch comprises a glass fiber and polyvinyl chloride master batch.

56. The process of claim 55 wherein the first master batch comprises about 10 to 20 wt. % glass fiber with the balance comprising a polyvinyl chloride polymer.

57. The process of claim 55 were in the second fiber comprises 35 to 40 wt. % of a wollastonite fiber.

58. The process of claim 54 wherein the first fiber master batch is a wollastonite fiber and a polyvinyl chloride polymer master batch.

59. The process of claim 58 wherein the master batch comprises about 30 to 55 wt. % of a wollastonite fiber with the balance comprising a polyvinyl chloride polymer.

60. The process of claim 54 wherein the second fiber comprises glass fiber.

61. The process of claim 56 were in the fiber in the first fiber master batch has 0.2 to 3 wt. % of an interfacial modifier coating.

62. The process of claim 57 wherein the second fiber has 0.2 to 3 wt. % of an interfacial modifier coating.

63. The process of claim 59 wherein the wollastonite fiber has 0.2 to 3 wt. % of an interfacial modifier coating.

64. The process of claim 60 wherein the glass fiber has 0.2 to 3 wt. % of an interfacial modifier coating.
